(19) United States
(12) Patent Application Publication
(10) Pub. No.: US 2005/0246802 A1
(43) Pub. Date: Nov. 3, 2005

(54) Bulk sorting of conifer somatic embryos

(76) Inventors: Stephen M. Attree, Victoria (CA); Irina Ivanovna Lobatcheva, Victoria (CA); Plamen Denchev, Victoria (CA); Reed Radley, Vancouver (CA); Lisheng Kong, Victoria (CA); Ming Shang, Victoria (CA)

Correspondence Address: KIRBY EADES GALE BAKER, BOX 3432, STATION D, OTTAWA, ON, K1P 6N9, CA

(21) Appl. No.: 10/999,918

(22) Filed: Dec. 1, 2004

Related U.S. Application Data
(60) Provisional application No. 60/526,007, filed on Dec. 2, 2003.

(51) Int. Cl. A01H 7/00; C12N 5/04

(52) U.S. Cl. 800/319; 435/419

(57) ABSTRACT

The selection and isolation of mature somatic conifer embryos has previously involved chemical based or labour intensive techniques. Disclosed are simple yet highly effective methods to sort somatic embryos according to maturity to generate a population of quality somatic embryos that exhibit a high probability of successful conversion and germination. The methods include liquid sorting of somatic embryos using an aqueous liquid having a specific solute concentration and/or a viscosity. In this way, embryos that have attained a degree of maturity (and preferably desiccation tolerance) can be separated both from embryos that are comparatively immature and importantly from significant quantities of proliferation material and other organic matter generated during embryo propagation.

### CROSS REFERENCE TO RELATED APPLICATION

This application claims the benefit of U.S. Provisional Application No. 60/526,007 filed Dec. 2, 2003 entitled “Bulk sorting of desiccation-tolerant conifer somatic embryos.”

### FIELD OF THE INVENTION

The present invention relates to the field of plant somatic embryos, their production and purification. In particular, the present invention relates to apparatuses and methods for separating conifer somatic embryos from proliferation matter and other organic material.

### BACKGROUND TO THE INVENTION

The development of somatic embryogenesis protocols for the clonal reproduction of plants has received considerable attention. Consequently, the specific steps of somatic embryogenesis have been documented in the art for a wide variety of plant species. All methods of somatic embryogenesis are known as tissue culture processes and generally commence with the selection of an explant from a desired plant. The explant is removed from the parent plant tissue by excision and is subsequently cultured on at least one medium to produce a cell mass capable of further differentiation and development. The cell mass can be maintained and proliferated in the undifferentiated state indefinitely, or manipulated to stimulate differentiation into mature somatic embryo structures which can be cultured further into mature somatic embryos (see for example U.S. Pat. Nos. 4,957,866; 5,238,835; 5,294,549; 5,491,090; 5,501,972; 5,563,061; 5,677,185, as well as PCT Publication No. WO96/37096, all of which are incorporated by reference). Matured somatic embryos can be harvested and germinated immediately, or dried and then germinated, or dried and stored until required for germination (for example, refer to U.S. Pat. Nos.: 5,183,835; 5,238,835, 5,413,930; 5,464,769, as well as PCT Publication No.: WO96/37095, all of which are incorporated by reference).

Proliferation and maturation of conifer somatic embryos must be conducted under controlled, sterile conditions. Protocols for the somatic embryogenesis of conifers typically involves several stages from initiation of embryogenic tissue through to somatic embryo maturation and germination. For initiation of conifer embryogenic cell lines whole gametophytes containing immature fertilized embryos or dissected immature fertilized embryos may be used as the explants. The explants may be placed in several different “initiation media” to initiate embryogenic tissue either with or without growth regulators. For maturation of conifer somatic embryos, initiated embryogenic cell lines are typically placed onto several media to maintain and develop the embryogenic tissue and multiply the number of embryos. Embryogenic tissue is then generally placed on a “maturation medium” to encourage the tissue to form mature embryos. Typically this maturation media contains abscisic acid (ABA). Methods for embryo proliferation and maturation are further described, for example, in U.S. Pat. No. 6,492,174, issued Dec. 10, 2002, and International Patent publication WO01/20972 published Mar. 29, 2001, which are also incorporated herein by reference.

Following the aforementioned steps of explant excision, explant culturing, and subsequent differentiation and development of somatic embryos, it is most desirable to select and isolate those embryos that have successfully completed the embryonic maturation process. Various techniques are known in the art for the evaluation of somatic embryo maturity.

Velho C. C., 1990 (Density Separation of Plant Embryos, MSc Thesis, Purdue University) proposed a mechanism for separation of mature embryos from immature embryos according to their relative densities. A decrease in embryo density was observed over a specific embryo maturation period. Moreover, following separation mature embryos exhibited an increased efficiency for conversion to seedlings compared to less mature embryos. Velho suggested a basic means to separate mature embryos from immature embryos according to relative buoyancy in uniform sucrose solutions having a predetermined density.

The production of somatic embryos can be very labour intensive. Typically, significant quantities of proliferation material and other miscellaneous organic matter are produced with either liquid or solid media-based culture systems. Before embryos are transferred for germination, or tested for maturity, they are manually extracted from this proliferation material. For this purpose, embryos are generally selected or ‘picked’ by hand from the copious proliferation material. The crude tissue culture products comprising the proliferation material are manually sifted, each selected embryo identified, individually grasped with tweezers, and separated from any adhered organic matter. This process is very labour intensive, and requires a skilled technician to differentiate the embryonic material from the proliferation material and other organic matter generated during the embryo development and maturation process.

It follows that there is a continuing need to develop methods and systems for the efficient selection and isolation of mature somatic embryos, in particular the isolation of mature conifer somatic embryos from proliferation material, that are suitable for continued propagation. Moreover, there is a need to develop corresponding methods that are suitable for large-scale identification, isolation, desiccation, and germination of mature embryos, without the need for labour intensive steps involving manual selection.

### SUMMARY OF THE INVENTION

It is an object of the present invention, at least in preferred embodiments, to provide a method for the selection of conifer somatic embryos that have achieved a predetermined degree of maturity.

It is a further object of the present invention, at least in preferred embodiments, to provide a method for the isolation of selected mature conifer somatic embryos, such that a high proportion of the isolated embryos are suitable for germination into seedlings.

It is a further object of the present invention, at least in preferred embodiments, to provide a method for the selection and isolation of high quality mature conifer somatic embryos.

It is a further object of the present invention, at least in preferred embodiments, to provide a method for the selection and isolation of high quality mature desiccation tolerant conifer somatic embryos.

It is a further object of the present invention, at least in preferred embodiments, to provide a means to separate mature plant somatic embryos from crude embryo compositions (including proliferation material), without the need for manual sifting and analysis of the compositions.

It is a further object of the present invention, at least in preferred embodiments, to provide desiccation-tolerant embryos.

The present invention provides significant improvements over the methods of the prior art for the separation of mature somatic conifer embryos. In selected embodiments the present invention also provides significant improvements and extensions over Velho, (1990).

Conifer somatic embryos undergo an alteration of triacylglycerol (TAG) content as they mature. The inventors have determined that the degree of embryo maturity correlates directly with the levels of TAG, and indirectly with the overall density of the embryo. Importantly, the inventors have recognized that this progressive reduction in embryo density can be effectively utilized as a ‘marker’ indicative that a particular embryo has successfully achieved a predetermined maturation stage. This enables the development simple, highly effective methods to isolate mature, viable embryos by virtue of their relative density.

Another important aspect of the present invention pertains to the discovery that sucrose solutions, and other solutions having equivalent densities, can be used not only to separate mature conifer somatic embryos from less mature embryos, but also to separate mature embryos from proliferation material and other organic matter generated during tissue culture procedures.

The inventors have established conditions for the efficient isolation of high quality, mature conifer somatic embryos. In preferred embodiments, such conditions involve for example the use of a specific gravity gradient such as a sucrose density gradient, optionally in combination with viscosity gradient. Such methods can give rise to a striking 5-fold increase in the ratio of high quality, viable embryos, to the total embryos present, whilst simultaneously removing almost all of the proliferation material. Using this method for spruce resulted in 95% conversion rates, with only a 5% loss of good embryos.

In a first aspect, there is provided a method of isolating a population of mature conifer somatic embryos from a crude embryo composition containing mature conifer somatic embryos, immature conifer somatic embryos, and proliferation material, the method comprising the steps of:
providing a body of aqueous liquid having density greater than that of said mature conifer somatic embryos, and less than that of said immature conifer somatic embryos and of said proliferation material;
immersing the crude embryo composition in the body of liquid;
permitting the components of the crude embryo composition to separate in the body of liquid in accordance with their densities relative to the liquid; and
removing the buoyant mature somatic conifer embryos from an upper portion of the body of liquid.

Preferably, the step of permitting comprises agitating or stirring the body of liquid to assist the components of the crude embryo composition to separate in the body of liquid in accordance with their densities relative to the liquid. Preferably, the stirring or agitating is effected by a mechanical stirring or agitation means. More preferably, the mechanical stirring means comprises a magnetic stir bar.

Preferably the body of liquid comprises a solution of one or more sugars, and optionally ABA. More preferably, the body of liquid comprises a 5% to a 25% solution of sucrose. More preferably, the body of liquid comprises a 16% to a 20% solution of sucrose. Most preferably, the body of liquid comprises an 18% solution of sucrose. Preferably, the body of liquid further comprises a viscosity enhancing agent. More preferably, the viscosity enhancing agent is selected from the group consisting of: transfergel, carboxymethylcellulose, hydroxymethylcellulose, and polysucrose. Preferably, the body of liquid comprises a density gradient, the components of the crude embryo composition separating according to their relative densities. Preferably, the body of liquid further comprises a viscosity gradient. More preferably, the viscosity gradient is generated by the presence of a viscosity enhancing agent selected from the group consisting of: transfergel, polysucrose, carboxymethylcellulose, and hydroxymethylcellulose. More preferably, the body of liquid comprises both a density gradient and a viscosity gradient. More preferably, the density gradient is a sucrose density gradient, and the viscosity gradient is a transfergel viscosity gradient, whereby the transfergel and a higher concentration of sucrose settle at lower portions of the body of liquid, and a lower concentration of sucrose exists at higher portions of the body of liquid.

Preferably, the mature somatic conifer embryos exhibit an increased level of triacylglycerol in comparison to the immature somatic conifer embryos.

In another aspect, the present invention provides for a method of isolating a population of mature conifer somatic embryos that exhibit tolerance to desiccation, from a crude embryo composition comprising mature conifer somatic embryos, immature conifer somatic embryos, and proliferation material, the method comprising the steps of:
(a) providing a body of aqueous liquid having a density greater than that of said mature conifer somatic embryos, and less than that of said immature conifer somatic embryos and said proliferation material;
(b) immersing the crude embryo composition in the body of liquid to produce a suspension;
(c) permitting the components of the crude embryo composition to separate in accordance with their relative densities;
(d) adding the suspension to a floatation solution, or adding a floatation solution to the suspension, the floatation solution having a density greater than that of said mature conifer somatic embryos and equal to or less than a density of said body of liquid, and optionally comprising ABA, the suspension at least initially forming a layer generally beneath the floatation solution;
(e) removing the buoyant mature somatic conifer embryos from an upper portion of the floatation solution;
wherein steps (c) and (d) may be performed in any order.

Preferably, the step of permitting comprises agitating or stirring the body of liquid to assist the components of the crude embryo composition to separate in the body of liquid in accordance with their densities relative to the liquid.

Preferably, the step of adding encompasses pouring the suspension from a first vessel into a second vessel, the second vessel containing the floatation solution, through an elongated funnel having a lower end extending near the bottom of the floatation solution, thereby to assist the suspension to at least initially form a layer generally beneath the floatation solution.

In another aspect, the invention provides for a method as previously described,, further comprising the step of:
adding a floatation solution to the body of liquid, or adding the body of liquid to a floatation solution, prior to the step of removing, the floatation solution optionally comprising ABA and having a density substantially greater than the density of said mature conifer somatic embryos, to lift the buoyant mature somatic conifer embryos to an uppermost portion of the body of liquid, thereby to facilitate isolation of the mature conifer somatic embryos.

In another aspect, the invention provides for a method of separating mature conifer somatic embryos from immature conifer somatic embryos and non-embryonic organic matter, the method comprising the steps of:
providing a crude embryo composition comprising mature conifer somatic embryos, immature conifer somatic embryos, and non-embryonic organic matter;
providing a body of liquid having specific gravity;
immersing the crude embryo composition in the body of liquid;
stirring or agitating the body of liquid and the immersed crude embryo composition, the mature embryos exhibiting a greater buoyancy than the immature embryos and the non-embryonic organic matter in the body of liquid; and
isolating the buoyant mature somatic conifer embryos from an upper portion of the body of liquid.

Preferably, in accordance with any of the methods of the present invention, the conifer somatic embryos are derived from a species selected from Douglas-fir and white spruce.

In another aspect, the invention provides for a composition comprising conifer somatic embryos isolated in accordance with any method of the present invention, the composition comprising at least 4 times the number of mature conifer somatic embryos suitable for germination, relative to the number of immature somatic embryos, the composition being substantially free of propagation material.

In another aspect, the invention provides for a mature somatic embryo derived from the composition of the present invention.

In another aspect, the invention provides for a conifer derived from the embryo of the present invention.

In another preferred aspect of the methods of the present invention, the mature somatic plant embryos exhibit a high degree of desiccation tolerance in comparison to the immature somatic plant embryos. The invention encompasses the methods described herein, further comprising the step of desiccating the isolated mature somatic conifer embryos, and desiccated embryos generated thereby.

In accordance with the present invention, any of the methods may further comprise the step of:
adding a floatation solution to the body of liquid, or adding the body of liquid to a floatation solution, prior to the step of removing, the floatation solution having a density substantially greater than the density of said mature conifer somatic embryos, to lift the buoyant mature somatic conifer embryos to an uppermost portion of the body of liquid, thereby to facilitate isolation of the mature conifer somatic embryos.

### BRIEF DESCRIPTION OF THE DRAWINGS

FIG. 1 provides a flow chart indicating the steps of a preferred method of the present invention, for separating mature conifer somatic embryos from a crude embryo composition.

FIG. 2 provides a comparative graph of the ratio of high quality mature embryos to total embryos either before or after a single round of sorting in accordance with the methods of the invention.

FIG. 3 illustrates the results of an experiment to determine the optimum sucrose concentration for the separation of conifer somatic embryos.

FIG. 4 illustrates comparative results of the average percentage of high quality mature somatic embryos to total somatic embryos, both prior to sorting, and from top and bottom fractions of a body of liquid after processing in accordance with the methods of the present invention.

FIG. 5 illustrates comparative results of the methods of the present invention, wherein different separation solutions are compared for sorting efficiency. Standard arrows are based upon 2-4 repetitions of each experiment.

FIG. 6 illustrates comparative results of the conversion rates (%) for germination of somatic embryos either directly germinated or germinated following sorting and desiccation in accordance with the methods of the invention.

FIG. 7 provides a microscope image of generally mature White Spruce embryos purified in accordance with the method of the present invention.

### DEFINITIONS

Body of liquid typically refers to an aqueous solution used for liquid sorting of mature embryos from immature embryos and proliferation material, if present. The body of liquid may comprise sugars such as sucrose in a concentration sufficient to make the density of the body of liquid more than that of mature somatic embryos, but less than that of less mature somatic embryos or proliferation material. Preferably, the body of liquid also comprises ABA (especially if the embryos will be desiccated prior to germination).
Bulk sorting the process of purification of a large amount of embryos at one time; a procedure designed for scale-up.
Conversion the ability of a somatic embryo to germinate (either in vitro or ex vitro) and subsequently develop into an established, autotrophic plant.
Crude embryo composition a composition of organic matter including somatic embryos (mature and immature), proliferation material generated during embryo production and maturation, and other organic and inorganic matter derived, for example from tissue culture processes. Typically, the crude embryo composition takes the form of a fairly dry, granular or fibrous mixture of the aforementioned components. The crude embryo composition may be derived from any suitable source of cultured somatic conifer embryos, including but not limited to bioreactor cultures, flask cultures, and petri plates.
Desiccation the drying of a somatic embryo by any means to a moisture content less than that of the original hydrated embryo.
Desiccation tolerant: refers to a preferred characteristic of mature, somatic conifer embryos isolated in accordance with the methods of the present invention. Such embryos may be desiccated following isolation, and reactivated by imbibition and germination. The tolerance to desiccation is reflected by the fact that most of such embryos are expected to subsequently undergo successful germination under appropriate conditions.
Floatation solution: solution sometimes used to ‘top-up’ a liquid culture or body of liquid comprising a crude embryo composition, prior to separation of the buoyant or floating mature plant somatic embryos from the upper portions of the culture. For example, the floatation solution may be added to a liquid culture or body of liquid comprising a crude embryo composition, to ‘lift’ the buoyant and floating embryos to the top of a vessel to facilitate their isolation by pouring off the uppermost portion of the liquid containing the mature embryos. Alternatively, the liquid culture or body of liquid comprising a crude embryo composition may be added to the floatation solution. Typically, a floatation solution may comprise about 16% sucrose and optionally ABA (especially if embryos are to be desiccated prior to germination).
Germination the initiation of physiological processes in a mature quiescent somatic embryo, induced by the uptake of water (imbibition) and exposure to inductive environmental cues, resulting in meristematic growth (cell division and elongation), and ending with the complete development of cotyledons.
Immature somatic embryo an embryo that is developmentally immature.
Incubation/Incubate relates generally to a step of holding or containing an item such as a cell/embryo culture at a desired fixed or variable temperature, which may be optional or preferred with respect to most of the embryo separation methods of the present invention. The step of incubation encompasses conditions of from 2-35° C., typically 20-25° C., more typically room temperature, and the incubation may last from 1 second to several hours.
Lipids One of a class of compounds which contain long-chain aliphatic hydrocarbons and their derivatives, such as fatty acids, alcohols, amines, amino alcohols, and aldehydes; includes waxes, fats, and derived compounds. Also known as lipin; lipoid.
Mature somatic embryo an embryo that has completed or substantially completed development from an internally-controlled dormancy (physiological) to an externally controlled dormancy (environmental) (E.G. quiescent dormancy), and can be readily germinated into a well-formed (plantable) germinant. This embryo has accumulated lipids or fatty acids to the point that its density is lowered and therefore can be immersed in purification solution and will exhibit a degree of buoyancy.
Polysucrose (Ficoll™) is a synthetic high molecular weight polymer made by the co-polymerization of sucrose and epichlorohydrin. The molecules have a branched structure with a high content of hydroxyl groups giving a good solubility in aqueous media.
Proliferation material Organic matter that is generated during proliferation and maturation of somatic embryos. The proliferation material is typically granular or fibrous.
Purification solution a liquid solution made up of sucrose at different concentrations, or of sucrose in addition with other chemicals, in which somatic embryos are immersed and separated, i.e. come into equilibrium based on density. The mature embryos that are less dense than the purification solution will exhibit a degree of buoyancy, or float to the top of the body of liquid.
Purification the act of removing non-embryonic tissue (suspensor, proliferative and undifferentiated embryonic tissues) from a batch of mature embryos at the end of the maturation phase (pre-germination purification)
Separate refers in specific embodiments to the separation of a crude embryo composition into mature somatic conifer embryos, immature somatic conifer embryos, and proliferation material. It should be noted that “separate” also encompasses “substantially separate”, since mature embryos may not be entirely separated from immature embryos and proliferation material each time the methods of the invention are utilized.
Somatic embryo a plant embryo formed in vitro from vegetative (somatic) cells by mitotic division of cells.
Specific gravity equivalent to relative density with particular reference to aqueous liquids, and the solute concentration therein.
Sucrose equivalent a density of sucrose solution equivalent to the embryo density.
Sucrose C₁₂H₂₂O₁₁ Combustible, white crystals soluble in water, decomposes at 160 to 186° C. A disaccharide of glucose and fructose.
TAG (triacylglycerol) Acylglycerols (glycerides) are fatty acid esters of the trihydroxy alcohol, glycerol. They are the main constituents of natural fats and oils. Triacylglycerols have all three of the glycerol hydroxyl groups esterified with fatty acids and are called triglycerides.
Transfergel hydroxyethylcellulose carrier gel of a highly viscous (almost syrupy) nature which does not thicken significantly after autoclaving.
Yield the amount, based on number, of mature somatic embyros captured as a result of purification or bulk sorting.
Zygotic embryo an embryo derived from the sexual fusion of gametic cells produced by meiosis, i.e. a plant seed.

### DETAILED DESCRIPTION OF THE PREFERRED EMBODIMENTS

Large-scale production of clonally derived plants, including conifers, depends increasingly upon automation. Any step that involves manual manipulation of plant embryos or seedlings can decrease the efficiency of cloned plant production, which ultimately effects the unit sale price of each plant. The present invention presents significant opportunities to improve the automation of somatic embryo production and germination, to generate autotrophic plants at lower cost.

The inventors of the present invention have further identified a key step in cloned conifer seedling production that is inefficient and labour intensive, namely the selection of mature conifer somatic embryos suitable for subsequent germination. Importantly, the inventors have recognized that this key step can be a rate-limiting factor in the propagation of cloned coniferous plants.

In general, the term “selection” includes both the identification of mature somatic embryos (compared to immature embryos), as well as the isolation of-such mature somatic embryos away from unwanted organic material, including immature embryos, proliferation matter and other organic debris. To date, such selection procedures have involved complex and/or labour intensive protocols in order to achieve a reasonable yield of high quality mature embryos, which are both suitable for germination, and substantially free of non-embryonic organic debris. The selection method should work for any conifer species that produces good quality embryos.

The propagation and development of mature somatic conifer embryos is a multi-step process involving the use of various media and growth factors. A significant amount of superfluous organic matter and debris can be generated during this process. Once the embryos have achieved a specific stage of maturity, they must be separated from this debris for subsequent germination. Previously, highly skilled technicians have undertaken this meticulous and laborious task. For this purpose, dishes comprising the embryos and the organic matter must be placed under a low-power light microscope, and sifted very carefully by hand. Once suitable mature conifer embryos have been identified within the debris, they must be individually grasped between tweezers, lifted from the dish and treated to remove any adhered debris. The embryos must then be transferred to another suitable location and growth media for germination.

In contrast to the prior art, the present invention encompasses a variety of related methods that enable fast and efficient selection and isolation of mature conifer somatic embryos substantially free of proliferation material. The methods are also relatively simple; they do not rely upon complex chemical testing, or meticulous manual inspection. Most importantly, the methods of the present invention are particularly suited for application on a large industrial scale. In this way, there is little or no requirement for manual selection of the embryos—the starting material can involve, for example, rather crude embryo compositions comprising a significant proportion of proliferation material and other miscellaneous organic matter, typical of conifer embryo production.

The invention relies in part upon the relative buoyancy of the components of the crude embryo compositions, and in particular the higher relative buoyancy of the mature somatic embryos as compared to the immature embryos, as well as the other organic material present. It also relies, at least in preferred embodiments, upon the viscosity of the sorting medium. The methods of the present invention circumvent the labour intensive steps of the prior art. In one important aspect, the present invention pertains to the realization that changes in conifer somatic embryo density occur as embryos progress towards maturity. This observation correlates with the previous observations of Velho (1990) with conifer zygotic embryos and somatic celery embryos. The present invention provides significant improvements over the work of Velho (1990) by the application of density separation techniques to conifer somatic embryos. Importantly, the methods of the present invention allow very crude embryo compositions to be separated, the crude embryo compositions comprising not only embryos, but also significant quantities of other organic matter, including but not limited to proliferation material generated during the culturing process. In this way, the present invention circumvents one or more highly labour intensive steps in conifer somatic embryo production, including the manual inspection and sifting of crude embryo compositions derived from culture media.

As discussed, the methods of the present invention provide a composition of mature, somatic conifer embryos that exhibit excellent germination characteristics. Further, such embryos also exhibit excellent tolerance to desiccation, as discussed in the Examples. For this reason, the methods of the invention, at least in preferred embodiments, allow for the production of mature somatic embryos amenable to desiccation and storage prior to subsequent imbibition and germination under appropriate conditions.

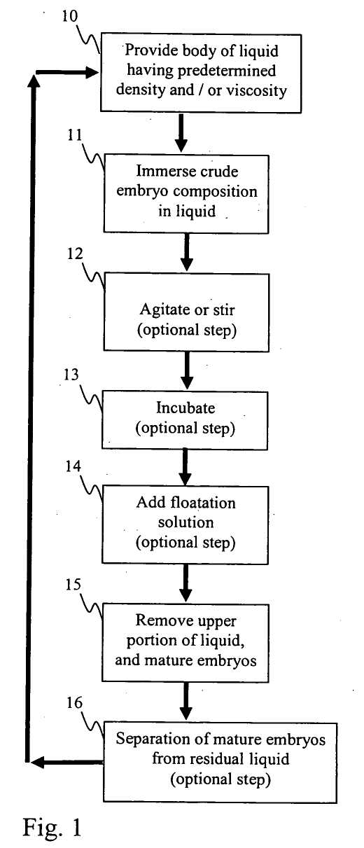

One preferred embodiment of the invention is illustrated with reference to FIG. 1. At step 10 there is provided a body of liquid having a predetermined specific gravity or relative density. The specific gravity of the liquid can be manipulated by varying the solute composition of the liquid. Typically, in preferred embodiments of the invention, the principle solute is sucrose. Sucrose solutions do not have a derogatory effect upon the metabolism of the plant (with the possible exception of affecting the osmotic potential of the plant cells). However, it should be noted that the present invention is not limited to the use of sucrose solutions. Any solute that can be dissolved to a sufficient degree may be utilized in accordance with the teachings of the present invention. Preferably, the solute is generally inert and non-toxic, having minimal effect upon embryo viability. If the embryos are to be subsequently desiccated, final washing sugars are preferred. At step 11, the crude embryo composition is immersed in the body of liquid. At step 12 the body of liquid and the immersed crude embryo composition are agitated and/or stirred in a manner suitable to break up any clumps of embryos and proliferation material. In this way, individual embryos may be separated from one another, and from adhered proliferation material.

Any form of stirring and/or agitation means may be used in accordance with the teachings of the present invention. Stirring via a mechanical stir bar pertains to particularly preferred method for breaking up any clumps of embryos and/or proliferation material, since such mechanical stirring is often aggressive enough to induce break-up of clumps without significant damage to the embryos. In any event, the break-up of any clumps of material induces the formation of a ‘suspension’ of material in the body of liquid.

Incubation step 13 may optionally be conducted prior to, simultaneously with, or following agitation/stirring step 12. Typically, the suspension generated in step 12 is incubated for a specific period of time sufficient to permit the components of the suspension to separate out according to their density relative to the liquid. If step 12 of agitation or stirring is conducted simultaneously with incubation step 13, then the agitation or stirring of the body of liquid may help to facilitate migration and settling but of the components of the crude embryo composition.

At optional step 14, the buoyant or floating mature somatic embryos may be further differentiated from the less mature embryos and proliferation material by the addition of a floatation solution. The floatation solution preferably has a density slightly less than the body of liquid used for sorting and greater than the average density of a mature embryo. In one embodiment, the floatation solution may be carefully added to the upper portion of the vessel, such that the floatation solution generally forms the uppermost portions of the body of liquid. In this way, the mature embryos may further migrate into the floatation solution and are lifted to the very top of the vessel to facilitate their removal from the vessel for example by pouring or siphoning at step 15. Another embodiment of the invention pertains to the transfer of the body of liquid containing the suspension to a second vessel containing a floatation solution. For example, in a particularly preferred embodiment of the invention, the suspension may be poured into the second flask via a long-neck funnel positioned such that the end of the spout of the funnel is located well below the meniscus of the floatation solution. In this way, the suspension will be induced to form a layer generally beneath the floatation solution. Subsequently, mature embryos will be induced to migrate upwards or float to the top of the floatation solution and well away from the less mature embryos and the proliferation material. The density of the body of liquid and the floatation solution are preferably similar: for example the body of liquid may have a density of 18% sucrose equivalent, and the floatation solution may have a density of 16% sucrose equivalent. In this way, both the floatation solution and the initial body of liquid have a density greater than the mature embryos, allowing them to migrate upwards accordingly. Solutions may contain absisic acid (ABA), particularly if embryos are subsequently desiccated. The concentration of ABA in the solutions is preferably at least as high as the final concentration in the maturation medium.

With reference again to FIG. 1, at optional step 16, the portion of the body of liquid comprising the mature embryos (e.g. siphoned or poured off from the vessel at step 15) is treated to substantially remove any residual liquid from the embryos. Any means of treatment may be used in accordance with the teachings of the present invention to remove the residual liquid. Typically, the upper portion of the body of liquid (containing the mature embryos) may be siphoned or poured off from the top of the vessel containing the liquid. The upper portions of the liquid may be directed through a filter paper connected to a vacuum pump. In this way, the solid components including the mature somatic embryos are retained on the filter paper. In contrast, the liquid is drawn through the filter paper and discarded.

The mature embryos obtained at step 15 may be in a form suitable for subsequent germination. However, additional treatment steps may be required as necessary. For example, less mature embryos, proliferation material, and other organic matter maybe carried over with the mature somatic embryos during the aforementioned steps. If this occurs, the method steps may be repeated for example using a body of liquid having the same or an alternative composition and density. Therefore, the invention further encompasses methods involving multiple rounds of liquid sorting to achieve a useful yield of mature somatic embryos.

Without wishing to be bound by theory, the liquid sorting methods of the present invention are considered suitable for use in the selection of many different types of conifer somatic embryos. For example, the liquid sorting methods are particularly useful in the separation of crude embryo compositions derived from Douglas Fir and White Spruce.

With reference again to FIG. 1, the crude embryo compositions can effectively be “dumped” into the body of liquid (for sorting) without any form of pre-processing. Once immersed, mature embryos are induced to migrate to the upper portions of the liquid, by virtue of their buoyancy. Indeed, some of the mature embryos may be so buoyant that they float at the top of the liquid. In direct contrast, less mature or immature embryos, and also a large portion of the remaining organic matter and debris, will be denser than the body of liquid, and tend to sink to lower portions of the liquid. In fact, the majority of the immature embryos and the debris may be dense enough to sink to the bottom of the vessel containing the liquid, well away from the mature buoyant embryos.

In preferred embodiments of the methods of the present invention, the migration of the various components of the crude embryo composition may be enhanced or facilitated by carefully mixing or agitation of the body of liquid. For example, the body of liquid may be shaken or stirred, although care should preferably be taken that the mixing is not so vigorous as to cause damage to the embryos. Preferably, the body of liquid may be mixed by gentle mixing means such as, for example, the bubbling of air, oxygen or an inert gas through the body of liquid, or careful mechanical stirring. Indeed, the bubbling of a gas through the body of liquid pertains to an alternative embodiment of the invention, which although less aggressive than mechanical stirring can achieve adequate break-up of clumps for some crude embryo compositions in some circumstances. Any type of mixing tends to assist in general separation of the embryos, and the prevention of clumping, and other unwanted adherence for example with the sides of the vessel containing the body of liquid. The inventors have noted that crude embryo compositions comprising a significant proportion of proliferation material are especially prone to clumping. Typically, the proliferation material is fibrous in nature, and may have a tendency to form webs of organic material in solution. Such webs may capture and retain somatic embryos, thereby preventing the embryos to separate out according to relative density in the solution. For this reason, the use of agitation means such as the bubbling of air represents a particularly preferred aspect of the invention.

In further embodiments of the present invention, the body of liquid may comprise a density gradient, rather than a uniform relative density throughout. For example, the body of liquid may comprise a density gradient, wherein the lower portions of the body of liquid may comprise a higher solute concentration, and the upper portions of the body of liquid may comprise a lower solute concentration. Therefore, the distance from the top of the liquid may be approximately proportional to the specific gravity of the liquid.

The density gradient will typically comprise a range of densities that are suitable for efficient separation of the plant material. For example, if a sucrose concentration of 18% is known to be suitable for the separation of embryos of a particular species, then the density gradient may be generated to include from 15% sucrose at upper portions of the body of liquid, to 25% sucrose in the lower portions. In this way, the mature embryos will be induced to migrate towards to upper regions of the body of liquid, above the portion that comprises about 18% sucrose. In contrast, the denser material, including the immature embryos, proliferation material and other organic matter will be induced to sink to the lower portions of the liquid, beneath the portion that comprises about 18% sucrose. In this way, the density gradient may effectively enhance the speed and efficiency of mature embryo separation.

In other preferred embodiments, the methods of the present invention may further involve the use of a viscosity gradient. In this way, the lower portions of the body of liquid are not only denser, but also more viscous than the upper portions of the body of liquid. Any suitable agents may be added to the body of liquid to generate the viscosity gradient. For example, typical agents may include, but are not limited to, Transfergel™, carboxymethylcellulose, hydroxymethylcellulose, polysucrose etc. Such viscosity enhancing agents are preferably inert, having little or no effect upon the metabolism of the embryos. In another embodiment, the sucrose gradient alone may be sufficient to provide both a density gradient and a viscosity gradient simultaneously, without the need for an additional viscosity enhancing agent.

Without wishing to be bound by theory, the use of a viscosity-enhancing agent may have the effect of further improving the speed and efficiency of mature embryo separation. Moreover, the use of a viscosity-enhancing agent may make the methods of the present invention particularly suited to the separation of mature embryos from exceptionally large quantities of immature embryos, proliferation material and organic debris. Without wishing to be bound theory, it is likely that the presence of some form of viscosity enhancing agent may protect or partly protect delicate plant embryos from damage by mechanical stirring or agitation. The inventor has experienced some problems in this regard through the use of mechanical stir bars when used inappropriately, which can damage embryos beyond the point of recovery.

Any method may be used to establish a density and/or viscosity gradient in the body of liquid intended for separation of the embryos. One method is described below. However, it should be noted that the methods described below need not necessarily involve a density and/or viscosity gradient, and would be useful with separation solutions having a generally uniform density and/or viscosity.

In one embodiment, a crude embryo composition which includes mature embryos, less mature embryos (which are less suited for conversion), and proliferation material, may be placed in a conical flask. In addition, the flask may include a magnetic stir bar. A body of liquid is then added to the flask, the body of liquid having a relatively high density and/or viscosity. The stir bar is then activated to stir the mixture and ensure substantially uniform distribution of the components of the crude embryo composition. The stir bar also helps to break up any clumps of embryos, or clumps of embryos adhered to proliferation tissue. The crude embryo composition in liquid suspension is transferred to a second flask. Importantly, the second flask already contains a floatation solution having a density and/or viscosity greater than that of the mature somatic embryos. The body of liquid (including the mixed crude embryo composition) is poured into the floatation solution via a funnel comprising an elongated spout that extends to the bottom of the second flask. This induces the relatively dense/viscous liquid comprising the crude embryo composition to adopt a position within the second flask beneath the floatation solution. Subsequent incubation of the resulting mixture and the crude embryo composition allows the less dense components of the crude embryo composition (including the mature embryos) to migrate upwards into the floatation solution. At the end of the incubation period, a large number of mature embryos will migrate to the top of the floatation solution in the flask. In contrast, the denser, less mature embryos and the proliferation material will reside in the lower portions of the flask, in accordance with their higher relative density. The proliferation material may have a tendency to float up with mature embryos, but are slowed or prevented due to the higher drag effect from the viscosity agent on this tissue, compared to mature somatic embryos which rise up more rapidly.

In further preferred embodiments, the methods of the present invention involve a substantially continuous flow of crude embryo composition into the body of liquid, and a substantially continuous flow of mature embryos, and unwanted immature embryos/other organic matter from the body of liquid. In this way, the body of liquid, and the systems interacting with the body of liquid, can be automated as fully as possible. Furthermore, by minimizing human contact with the body of liquid, the sterility of the conditions can be substantially maintained.

In further preferred embodiments of the present invention, the body of liquid further comprises one or more preservatives to prevent unwanted microbial growth. Such preservatives may be selected from any soluble preservatives, fungicides, or microbicides that are known in the art, including those commonly used for tissue culture purposes.

Further embodiments of the invention will be discussed with reference to the following Examples, which are in no way intended to limit the scope of the invention disclosed and claimed:

### EXAMPLES

### Example 1

### Douglas-Fir Somatic Embryo Density is Correlated with TAG Content

Douglas-fir somatic embryos derived from four different cell lines were separated using a sucrose density gradient, in accordance with the methods of the present invention.

The results of these experiments are summarized in Table 1. For each of the cell lines tested, the top fraction of mature embryos consistently comprised higher levels of TAG when compared to the bottom fraction of immature embryos. On average, the top fraction of embryos comprised more than 30% higher levels of TAG compared to the bottom fraction of embryos in selected samples.

TABLE 1
Concentration of TAG in purified Douglas-fir somatic embryos
(total TAGs per mg fresh weight, ukg/mg).
TAG concentration in purified DF SE
(tiotal TAGs per mg of FW, ukg/mg)

| | | | | Soluble | Insoluble |
|---|---|---|---|---|---|
| Cell | | SE mor- | | protein, | protein, |
| line | Fraction | phology | TAGs | ukg/mg | ukg/mg |
| DF 7-5513 | Bottom | Abnormal | 181 | 87 | 29 |
| ″ | Bottom | Good | 204 | 68 | 21 |
| Mean | | | 193 | 78 | 25 |
| ″ | Top | Abnormal | 262 | 126 | 49 |
| ″ | Top | Good | 278 | 134 | 31 |
| Mean | | | 270 | 130 | 40 |
| DF 3-5724 | Bottom | Abnormal | 222 | 112 | 20 |
| ″ | Bottom | Good | 242 | na | na |
| Mean | | | 232 | 112 | 20 |
| DF 3-5724 | Top | Abnormal | 266 | 133 | 24 |
| ″ | Top | Good | 287 | 147 | 19 |
| Mean | | | 277 | 140 | 22 |
| DF-7-6559 | Bottom | | 99 | 45 | 12 |
| ″ | Top | | 143 | 65 | 13 |
| DF 7-5777 | Bottom | | 94 | 42 | 13 |
| ″ | Top | | 138 | 50 | 13 |
| Avr/line | Bottom | | 174 | 71 | 19 |
| | Top | | 229 | 109 | 25 |

These results indicate that TAG levels correlate inversely with embryo density.

### Example 2

### Optimization of Sucrose Density for Isolating Mature Douglas-Fir Somatic Embryos

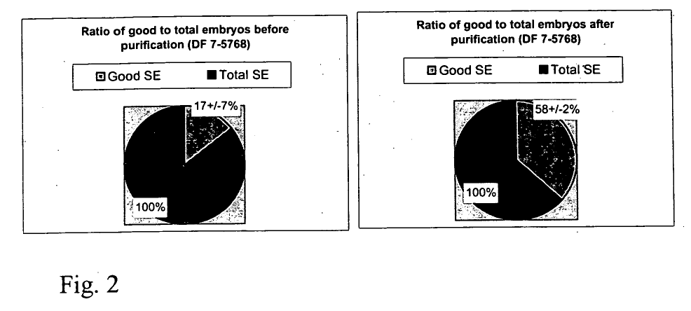

FIG. 2 illustrates typical results from the methods of the present invention. The experiments were conducted using Douglas-fir somatic embryos derived from bioreactors. The suitability of the embryos for germination was assessed on the basis of morphology. Prior to separation and isolation of the embryos, only about 17% of the embryos pertained to those that were deemed viable and suitable for germination. After purification, the total number of embryos was reduced. However, the proportion of those that were viable and suitable for germination jumped nearly 4-fold to more than 58%. Therefore, the selection methods of the present invention successfully excluded those immature embryos that are unsuitable for germination, whilst retaining a large share of mature ‘good’ embryos.

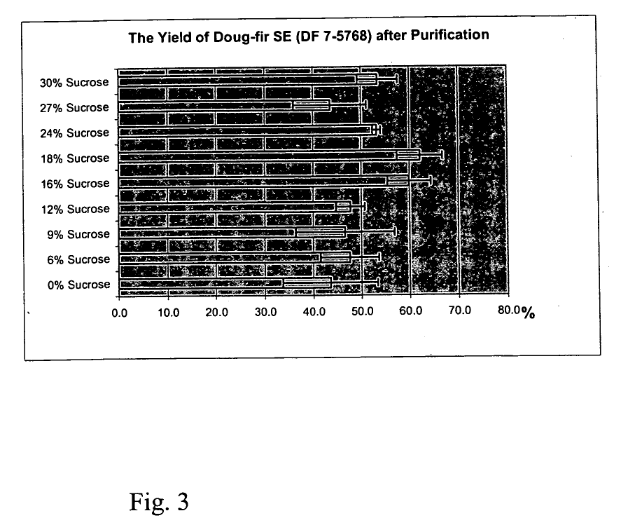

Further experimentation using Douglas-fir somatic embryos indicated that optimal separation conditions may require a body of liquid having a solute concentration equivalent to about 18% sucrose. Parallel experiments were conducted to immerse crude embryo compositions comprising Douglas-fir embryos into various bodies of liquid, each having a different solute concentration. Subsequently, the various components of the crude embryo compositions were allowed to separate within the body of liquid according to their relative densities. The solid material (mostly comprising embryos) was then isolated from the top fraction, and the embryos were processed for germination. FIG. 3 illustrates the percentage yield of high quality, mature embryos assessed on the basis of morphology. The highest percentage yield was observed for purification methods involving 18% sucrose, with generally lower yields observed for decreasing sucrose concentrations of 12, 9, 6, and 0% sucrose. Raising the sucrose concentration to 30% did not produce any significant increase in the percentage yield, and in fact lower yields were observed for 24 and 27% sucrose.

### Example 3

### The Use of a Viscosity Enhancing Agent in the Recovery of High Quality Mature Douglas-Fir Somatic Embryos

Somatic embryos of a Douglas-fir cell line (DF-7F) were used in the following experiment. The somatic embryos were divided into several groups for parallel experiments. A control group of embryos were desiccated and germinated without any form of bulk sorting. Further groups underwent bulk sorting procedures in accordance with the methods of the present invention, prior to desiccation and germination. In this way, the capacity of the bulk sorting methods of the present invention, to improve the yield of high quality mature embryos, could be directly tested and compared to a control embryo population.

The experiments were conducted in accordance with Bioreactor standard operating procedures, wherein the crude embryo extract was treated using the following solutions:
Purification treatment I—18% sucrose solution
Purification treatment II—18% sucrose+0.22% transfergel
Purification treatment III—18% sucrose+0.22% transfergel+7% polysucrose

Purification treatments took place in 0.51 flasks at room temperature with mechanical stirring.

After treatment with the purification solutions, the crude embryo extracts were treated with the following floatation solutions:
For purification treatments I and II (above)—16% sucrose
For purification treatment III—18% sucrose+4% polysucrose

For each treatment regime, the upper portion of floating embryos was isolated, desiccated and maturity assessed on the basis of morphology. The efficiency of isolation of mature embryos was compared to a control group of embryos derived from tissue culture without liquid sorting.

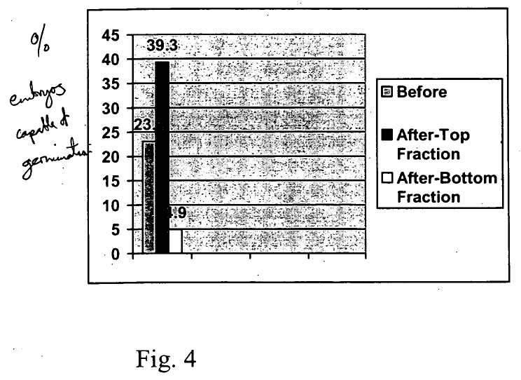

FIG. 4 illustrates the overall effectiveness of the methods of the present invention to separate the high quality mature embryos from the immature embryos (average results shown of several experiments). Prior to separation, 23.1% of the embryos were capable of successful germination. After separation, the top fractions of buoyant embryos exhibited a 39.3% yield—a significant increase. In contrast, the bottom fractions exhibited a very low yield of embryos capable of germination—only 4.9% of the total. This experiment demonstrates that separation of the embryos into top and bottom fractions by liquid sorting provides an effective means of improving the yield of mature, high quality embryos.

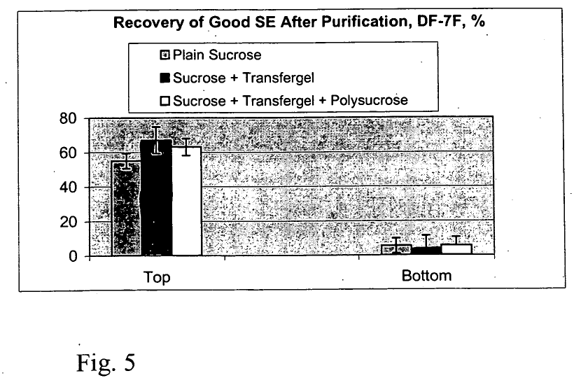

FIG. 5 provides a direct comparison of the capacity of the various purification solutions to induce efficient separation of high quality mature, and immature embryos. Purification with transfergel in the separation solution appeared to give the highest yield of embryos capable of subsequent germination (67% in the top fraction). Experiments were conducted using Douglas-fir somatic embryos, derived from bioreactors according to standard operating procedures, which underwent liquid sorting at room temperature. The maturity of the embryos was assessed on the basis of morphology.

### Example 4

### Purification and Desiccation of Douglas-Fir Somatic Embryos and the Effect on Yield

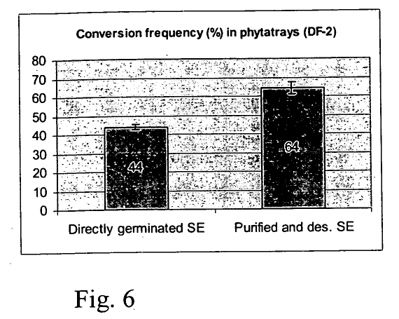

Purification of Douglas-fir somatic embryos in solutions containing 80 μM ABA and then desiccation resulted in the average quality of the germinants increasing. FIG. 6 illustrates a comparison of the conversion frequency (%) between directly germinated somatic embryos, and those which have been both purified in accordance with the methods of the present invention, and desiccated prior to germination. An increase from 44% conversion to 64% conversion frequency was observed.

### Example 5

### Visual Comparison of the Solid Matter Recovered from the Top and Bottom Fraction Following Bulk Sorting in Accordance with the Methods of the Present Invention

Upon visual inspection, the solid matter from the top fraction comprises almost exclusively a white granular matter, wherein each granule generally pertains to a somatic embryo. In direct contrast, the solid matter recovered from the bottom fraction includes somatic embryos (presumably mostly immature embryos—white matter), as well as a substantial proportion of darker proliferation matter and other organic debris. In summary, the processing of the crude embryo composition in accordance with the teachings of the present invention enables successful recovery of a fraction of the solid matter that consists primarily of useful plant embryos.

### Example 6

### A Two-Step Purification Method and its Effect on Yield of Mature Douglas-Fir Somatic Embryos

A series of experiments was conducted to assess a two-step method of liquid bulk-sorting of Douglas-fir somatic embryos. The somatic embryos (Douglas-fir line DF-7A) were taken from bioreactors at the end of the maturation process.

The somatic embryos were matured in bioreactors according to bioreactor standard operating procedures, using a body of liquid comprising 18% sucrose+0.22% transfergel for separation, followed by 16% sucrose+80 μM ABA (for floatation). The purified somatic embryos were then stored overnight at 4° C. and purified again by re-suspension in 20% sucrose+80 μM ABA. After the second purification step, the top fraction of the body of liquid was removed, and the embryos isolated therefrom. The embryos were desiccated and then germinated to assess their conversion rate. The conversion rates were compared with the conversion rate for corresponding control embryos germinated directly without purification or desiccation.

The results of the experiment are shown in Table 2. Approximately 68% of the embryos were carried through the two-step purification procedure and retained in the final preparation. However, the overall ratio of mature to total somatic embryos increased from only 20.3% (control embryos) to 57.7% (embryos after two-step purification). In summary, the two-step purification procedure appeared even more effective than the one-step procedures, at removing immature and abnormal embryos from the final fraction of solid material. Each purification step incurs some loss of embryos, but the ratio of high quality mature embryos that are suitable for germination increases significantly.

TABLE 2
Ratio and yield of mature and total Douglas-fir
somatic embryos after two-step bulk sorting

| | | After Two-step |
|---|---|---|
| Parameter | Before Purification | Purification |
| Ratio of Mature to Total | 20.3 +/− 1.2 | 57.7 +/− 1.4 |
| Somatic Embryos, % | | |
| Yield in Comparison to | 100 | 68.2 +/− 0.25 |
| Direct Germinated | | |
| Somatic Embryos, % | | |

The present invention therefore encompasses methods for the separation of high quality mature somatic embryos from immature embryos and/or other organic matter and debris, which involves one or more purification cycles, each cycle involving the use of a body of liquid having a predetermined solute concentration.

### Example 7

### Purification of White Spruce Somatic Embryos

Additional experiments were conducted using somatic embryos derived from white spruce. Methods for preparing spruce were described in U.S. Pat. No. 6,627,441 (incorporated herein by reference). When ABA was included in solutions desiccation tolerance was improved. The corresponding embryos were produced on Petri plates, and induced to undergo liquid sorting in accordance with the methods of the present invention. The body of liquid for liquid sorting comprised 18%, sucrose, 4% Ficoll and 0.22% transfergel. The concentration of the floatation solution was 18% sucrose+40 μM ABA.

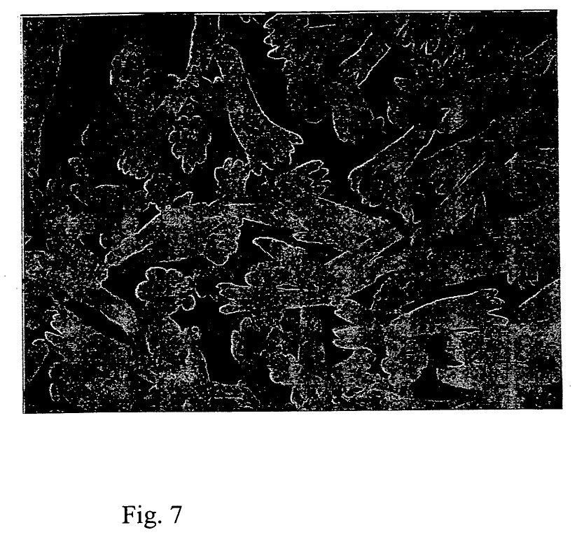

FIG. 7 provides a microscope image of the mature white spruce embryos-derived from the experiment, the majority of which exhibit a mature-type morphology. Little or no contaminant organic debris or proliferation material is present. Embryos were rinsed in 18% sucrose and 60 μM ABA. Embryos were then desiccated to about 5% moisture content.

Subsequent germination of the embryos derived from these experiments resulting in 95% conversion efficiency, which demonstrates the exceptional capacity of the methods of the present invention to provide high quality, mature embryos that are excellent for germination. The method resulted in only 5% loss of good embryos.

These experiments demonstrate the importance of the methods of the present invention for the purification of mature somatic embryos derived from a wide variety of coniferous species.

Whilst the present invention has been described with reference to specific examples and embodiments, these are in no way intended to be limiting, and are provided merely to demonstrate the capacity of the methods of the present invention to efficiently recover a population of high quality, mature somatic conifer embryos. It will be apparent to those skilled in the art upon a reading and understanding of the foregoing that methods other than the specific embodiments illustrated are attainable, which nonetheless lie within the spirit and scope of the present invention. It is intended to include all such methods, equivalents thereof, and products thereof within the scope of the appended claims.

1. A method of isolating a population of mature conifer somatic embryos from a crude embryo composition containing mature conifer somatic embryos, immature conifer somatic embryos, and proliferation material, the method comprising the steps of:
providing a body of aqueous liquid having density greater than that of said mature conifer somatic embryos, and less than that of said immature conifer somatic embryos and of said proliferation material;
immersing the crude embryo composition in the body of liquid;
permitting the components of the crude embryo composition to separate in the body of liquid in accordance with their densities relative to the liquid; and
removing the buoyant mature somatic conifer embryos from an upper portion of the body of liquid.

2. The method of claim 1, wherein the step of permitting the components to separate comprises agitating or stirring the body of liquid to assist the components of the crude embryo composition to separate in the body of liquid in accordance with their densities relative to the liquid.

3. The method of claim 1, wherein the body of liquid comprises a solution of one or more sugars.

4. The method of claim 3, wherein the body of liquid comprises a 5% to a 25% solution of sucrose.

5. The method of claim 4, wherein the body of liquid comprises a 16% to a 20% solution of sucrose.

6. The method of claim 5, wherein the body of liquid comprises an 18% solution of sucrose.

7. The method of claim 1, wherein the body of liquid further comprises a viscosity enhancing agent.

8. The method of claim 7, wherein the viscosity enhancing agent is selected from the group consisting of: transfergel, carboxymethylcellulose, hydroxymethylcellulose, and polysucrose.

9. The method of claim 1, wherein the body of liquid comprises a density gradient, the components of the crude embryo composition separating according to their relative densities.

10. The method of claim 1, wherein the body of liquid further comprises a viscosity gradient.

11. The method of claim 10, wherein the viscosity gradient is generated by the presence of a viscosity enhancing agent selected from the group consisting of: transfergel, polysucrose, carboxymethylcellulose, and hydroxymethylcellulose.

12. The method of claim 10, wherein the body of liquid comprises both a density gradient and a viscosity gradient.

13. The method of claim 12, wherein the density gradient is a sucrose density gradient, and the viscosity gradient is a transfergel viscosity gradient, whereby the transfergel and a higher concentration of sucrose settle at lower portions of the body of liquid, and a lower concentration of sucrose exists at higher portions of the body of liquid.

14. The method of claim 2, wherein the stirring or agitating is effected by a mechanical stirring or agitation means.

15. The method of claim 14, wherein the mechanical stirring means comprises a magnetic stir bar.

16. The method of claim 1, wherein the mature somatic conifer embryos exhibit an increased level of triacylglycerol in comparison to the immature somatic conifer embryos.

17. A method of isolating a population of mature, desiccation tolerant conifer somatic embryos, from a crude embryo composition comprising mature conifer somatic embryos, immature conifer somatic embryos, and proliferation material, the method comprising the steps of:
(a) providing a body of aqueous liquid having a density greater than that of said mature conifer somatic embryos, and less than that of said immature conifer somatic embryos and said proliferation material;
(b) immersing the crude embryo composition in the body of liquid to produce a suspension;
(c) permitting the components of the crude embryo composition to separate in accordance with their relative densities;
(d) adding the suspension to a floatation solution, or adding a floatation solution to the suspension, the floatation solution having a density greater than that of said mature conifer somatic embryos and equal to or less than a density of said body of liquid, and optionally comprising ABA, the suspension at least initially forming a layer generally beneath the floatation solution;
(e) removing the buoyant mature somatic conifer embryos from an upper portion of the floatation solution;
wherein steps (c) and (d) may be performed in any order.

18. The method of claim 17, wherein the step of permitting comprises agitating or stirring the body of liquid to assist the components of the crude embryo composition to separate in the body of liquid in accordance with their densities relative to the liquid.

19. The method of claim 17, wherein the step of adding encompasses pouring the suspension from a first vessel into a second vessel, the second vessel containing the floatation solution, through an elongated funnel having a lower end extending near the bottom of the floatation solution, thereby to assist the suspension to at least initially form a layer generally beneath the floatation solution.

20. The method of claim 1, further comprising the step of:
adding a floatation solution to the body of liquid, or adding the body of liquid to a floatation solution, prior to the step of removing, the floatation solution optionally comprising ABA and having a density substantially greater than the density of said mature conifer somatic embryos, to lift the buoyant mature somatic conifer embryos to an uppermost portion of the body of liquid, thereby to facilitate isolation of the mature conifer somatic embryos.

21. A method of separating mature conifer somatic embryos from immature conifer somatic embryos and non-embryonic organic matter, the method comprising the steps of:
providing a crude embryo composition comprising mature conifer somatic embryos, immature conifer somatic embryos, and non-embryonic organic matter;
providing a body of liquid having specific gravity;
immersing the crude embryo composition in the body of liquid;
stirring or agitating the body of liquid and the immersed crude embryo composition, the mature embryos exhibiting a greater buoyancy than the immature embryos and the non-embryonic organic matter in the body of liquid; and
isolating the buoyant mature somatic conifer embryos from an upper portion of the body of liquid.

22. A method of claim 1, wherein the conifer somatic embryos are derived from a species selected from Douglas-fir and white spruce.

23. The method of claim 1, further comprising the step of: desiccating the mature somatic conifer embryos.

24. A composition comprising conifer somatic embryos isolated in accordance with the method of claim 1, the composition comprising at least 4 times the number of mature conifer somatic embryos suitable for germination, relative to the number of immature somatic embryos, the composition being substantially free of propagation material.

25. A mature somatic embryo derived from the composition of claim 24.

26. A conifer derived from the embryo of claim 25.

27. A method of claim 17, wherein the conifer somatic embryos are derived from a species selected from Douglas-fir and white spruce.

28. The method of claim 17, further comprising the step of: desiccating the mature somatic conifer embryos.

29. A composition comprising conifer somatic embryos isolated in accordance with the method of claim 17, the composition comprising at least 4 times the number of mature conifer somatic embryos suitable for germination, relative to the number of immature somatic embryos, the composition being substantially free of propagation material.

30. A mature somatic embryo derived from the composition of claim 29.

31. A conifer derived from the embryo of claim 30.

32. A method of claim 21, wherein the conifer somatic embryos are derived from a species selected from Douglas-fir and white spruce.

33. The method of claim 21, further comprising the step of: desiccating the mature somatic conifer embryos.

34. A composition comprising conifer somatic embryos isolated in accordance with the method of claim 21, the composition comprising at least 4 times the number of mature conifer somatic embryos suitable for germination, relative to the number of immature somatic embryos, the composition being substantially free of propagation material.

35. A mature conifer somatic embryo derived from the composition of claim 34.

36. A conifer derived from the embryo of claim 35.

37. The method of claim 1 wherein the body of liquid comprises ABA.
